(19) United States
(12) Patent Application Publication
(10) Pub. No.: US 2026/0071230 A1
(43) Pub. Date: Mar. 12, 2026

(54) Precise Bioengineering Of Flowering Time For Bioenergy Production And Gene Containment

(71) Applicant: Brookhaven Science Associates, LLC, Upton, NY (US)

(72) Inventors: Meng Xie, Yaphank, NY (US); Dimiru Tadesse, Frederick, MD (US)

(21) Appl. No.: 19/064,919

(22) Filed: Feb. 27, 2025

Related U.S. Application Data
(60) Provisional application No. 63/558,606, filed on Feb. 27, 2024.

(51) Int. Cl. C12N 15/82; C07K 14/415

(52) U.S. Cl. CPC C12N 15/827; C07K 14/415

(57) ABSTRACT

Various examples relate to a transgenic crop plant where a sequence of a cis-regulatory element in the promoter of certain genes is genomically changed in order to alter the flowering time or flowering rate of the plant. In one example, the cis-regulatory element in the promoter of such certain genes may be modified from TGAATG(A/T) (A/T/C) to TGAGGG(A/T) (A/T/C). The certain genes may be genes that encode SbEhd1, OsFTL10/FT8, SbFT10, ZmELF3.2, or combinations thereof. And, the crop plant can be for example, sorghum, maize or rice. In another example, the present transgenic crop plant is a progeny plant. In yet another example, flowering time of the transgenic crop plant is regulated by a method that includes changing the sequence of the cis-regulatory element in the promoter of certain genes.

### CROSS-REFERENCE TO RELATED APPLICATIONS

This application is a Non-Provisional application which claims benefit of U.S. Patent Provisional Application No. 63/558,606, filed on Feb. 27, 2024, which is herein incorporated by reference in its entirety.

### STATEMENT OF GOVERNMENT RIGHTS

The present application was made with government support under contract number DE-SC0012704 awarded by the United States Department of Energy. The United States government has certain rights in the invention(s).

### REFERENCE TO A SEQUENCE LISTING

This application contains a Sequence Listing in computer readable form. The computer readable form is incorporated herein by reference. The computer readable file is named “IP2024-013-01_Sequence.xml” and was created on Jul. 27, 2025 (42,299 bytes).

### TECHNICAL FIELD

The present disclosure relates to the field of genetic manipulation of flowering to optimize biomass and food production of transgenic plants by controlling sorghum flowering time, specifically the manipulation of sorghum SbGhd7 binding activity to regulate floral transition by regulating genes for flowering activation and controlling sorghum flowering time.

### BACKGROUND

Cereal crops, maize, rice and sorghum are among the 15 crops that provide 90% of the world's food. In addition, sorghum is a viable biomass feedstock for bioenergy production due to its high photosynthesis efficiency and stress resilience. Floral transition or flowering is a trait that controls the switch between seed production (reproductive growth) and biomass production (vegetative growth). For food production, there is a preference by some for earlier flowering so the plants can be harvested multiple times in one year. However, for bioenergy production, later flowering may be preferred so that plants have more time to grow and accumulate biomass in stems and leaves.

Biological processes such as flowering depend on gene expression programs that involve the transcriptional regulation of thousands of genes. Transcriptional regulation depends on the recognition of specific promoter sequences, called cis-regulatory elements, by regulatory proteins also known as transcription factors (TFs). The binding between TF and cis-regulatory elements is a major determinant of the target specificity of TF. TFs and their associated genes form gene regulatory networks (GRNs) that are fundamental for controlling biological processes in plants. In plants, TFs account for only 5-10% of all genes that require transcriptional control. Considering that plants in general have tens of thousands of protein-coding genes (e.g. about 34,000 genes in sorghum), each plant TF controls hundreds and even thousands of different genes.

In cereal crops, flowering is induced by mobile florigens produced in the leaves. The expression or production of mobile florigens in leaves is controlled by a GRN that contains multiple key and cereal crop-specific TFs, including GRAIN NUMBER, PLANT HEIGHT AND HEADING DATE 7 (Ghd7) and EARLY HEADING DATE 1 (Ehd1) 1. Molecular genetic understanding of flowering time regulation provides information for sorghum development. GRAIN NUMBER, PLANT HEIGHT AND HEADING DATE 7 (SbGhd7) is one of six classical loci conferring photoperiod sensitivity of sorghum flowering. However, there remains a need to study its functions.

Meanwhile, commercially growing transgenic and GMO crops are subject to US regulations due to concerns about gene flow to their sexually compatible wild weedy relatives. The manipulation of flowering especially flowering inhibition is a solution; but previous attempts have focused on overexpressing or knocking out transcription factors that control flowering time. Although flowering time was altered by this strategy, there were adverse side effects. For example, manipulations of earlier flowering were associated with hormonal disruptions, growth penalties and a reduction in seed number and size. These side effects appear to be unavoidable due to the wide range of transcription factor targets. Each plant transcription factor generally targets hundreds of genes involved in various biological pathways. Therefore, traditional methods are problematic due to the inevitable effects on all targets of the transcription factor.

To solve this problem, a strategy that can precisely manipulate the regulatory association between transcription factor and desired target genes is needed, for example, by modifying a specific cis-regulatory element.

### SUMMARY

The present disclosure is a short DNA sequence (cis-regulatory element) TGAATG(A/T)(A/T/C) that is distributed in promoters of certain flowering genes and may be the target for manipulating flowering time of sorghum, a bioenergy and food crop by altering the cis-regulatory element.

This cis-regulatory element is recognized and bound by a strong flowering repressor factor in sorghum called SbGhd7, overexpression of which substantially or completely blocks flowering. And in rice and maize the SbGhd7 homologue is called Ghd7. SbGhd7 from sorghum and the Ghd7 proteins from rice and maize have sequence similarity. Therefore, rice and maize Ghd7s may bind to the same cis-regulatory element as sorghum SbGhd7. It has been found in vitro studies that changing the sequence of this cis-regulatory element (e.g., changing TGAATGAA to TGAGGGAA) eliminates SbGhd7 binding, providing an opportunity to unwire SbGhd7 and its target genes to precisely reprogram the gene regulatory network that governs sorghum flowering time. In addition, it has been found that this cis-regulatory element is also present in promoters of flowering genes or key flowering genes in maize and rice, suggesting that the cis-regulatory element in maize and rice may be targets for manipulating flowering time of maize and rice or other species with sequence similarity.

The advantage of this technique is the ability to manipulate the gene regulatory network regulated either directly or indirectly by SbGhd7/Ghd7 precisely and with minimal side effects. Each plant transcription factor generally targets hundreds of genes involved in various biological pathways. The traditional methods that manipulate transcription factor expression are problematic due to the inevitable effects on all gene targets regulated either directly or indirectly by the transcription factor. The present method of removing, mutating or editing the cis-regulatory element in the promoter of desired gene(s) provides a more precise way to reprogram the flowering gene regulatory network regulated either directly or indirectly by SbGhd7/Ghd7 with much fewer side effects.

The molecular functions of SbGhd7 were characterized. The gene regulatory network controlled by SbGhd7 was constructed and validated. The biological roles of SbGhd7 and some of its major targets were studied. The net result of preventing flowering is to increase the length of the growth season which results in increased biomass accumulation, while the net result of accelerating flowering is to increase grain yield.

SbGhd7 overexpression completely prevented sorghum flowering. Additionally, we show that SbGhd7 is a major negative regulator of flowering, binding to the promoter motif TGAATG(A/T)(A/T/C) and represses the transcription of the major florigen FLOWERING LOCUS T 10 (SbFT10) and floral activators EARLY HEADING DATE (SbEhd1), FLAVIN-BINDING, KELCH REPEAT, F-BOX1 (SbFKF1) and EARLY FLOWERING 3 (SbELF3).

Studies related to the present disclosure suggest that SbGhd7 is a repressor of sorghum flowering by directly and indirectly targeting genes for flowering activation. In the examples studied, SbGhd7 directly represses the activity of SbFKF1, SbELF3 and SbFT10 while indirectly targeting the activity of SbFT1 or SbFT8 other regulators.

The present technique is to precisely manipulate crop flowering time by modifying the cis-regulatory element TGAATG(A/T)(A/T/C). In contrast to prior art strategy(ies), the present approach of removing, editing or mutating the cis-regulatory element in the promoter of desired target genes modulated either directly or indirectly by SbGhd7/Ghd7 may provide a more precise way to reprogram the flowering with fewer or much fewer side effects.

Previous studies suggest that this cis-regulatory element plays an essential and conserved role in regulating flowering gene expression in crops. The cis-regulatory element that binds to sorghum SbGhd7 was identified in the present study. SbGhd7 has been shown to be a major flowering repressor in sorghum and its overexpression completely blocks flowering. SbGhd7 binds specifically to a cis-regulatory element present in promoters of certain target genes it regulates. Changing the sequence of the cis-regulatory sequence (e.g. changing TGAATGAA to TGAGGGAA) completely removed SbGhd7 binding. Furthermore, this cis-regulatory element appears to have broad regulatory effects on numerous flowering target genes, as it is widely present in promoters of these genes in sorghum, maize and rice.

Therefore, modification of this cis-regulatory element (e.g. removal or mutation) provides a novel tool to precisely impose or remove SbGhd7/Ghd7 repression on desired target gene(s) for obtaining a desired crop performance in food production and biomass production for bioenergy. In one embodiment, the present transgenic crop plant comprises a genomically changed sequence of a cis-regulatory element in the promoter of genes which alters the flowering time of the plant. Further with the present method the flowering time of a transgenic crop plant may be regulated by changing (e.g., removing or mutating) the sequence of a cis-regulatory element in the promoter of certain genes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1. —SbGhd7 overexpressing sorghum plants show delayed flowering phenotypes. SbGhd7 overexpressing sorghum plants represses the expression of genes that activate flowering. Quantitative RT-PCR results showing SbGhd7 overexpression represses the expression of SbFT1, SbFT8, SbFT10 and SbEhd1. Gene expression was normalized against SbActin, with Ctrl set as 1. Values represent means+SE, n=4. P values were calculated by two-tailed Student's t-tests.

FIGS. 2A-2F: SbGhd7 directly represses sorghum SbFT10, SbEhd1, SbFKF1 and SbELF3. FIG. 2A—SbGhd7 subcellular localization in sorghum protoplasts. Yellow fluorescence protein tagged SbGhd7 (SbGhd7-YFP; green) and the nuclear marker (mCherry-VirD2NLS; magenta) were coexpressed in protoplasts and their fluorescent signal overlap is indicated in yellow. Scale bar: 5 μm; FIG. 2B—transient transactivation assay for SbGhd7 repressor activity. Top scheme displays the four types of vectors used. The transactivator construct overexpressing herpes simplex virus VP16 transactivator fused with the LexA DNA-binding domain (LBD) was used to constitutively activate GUS expression through the interaction between LexA and LBD; FIG. 2C—transient transactivation assay testing SbGhd7 transcriptional repression on SbEhd1 promoter. Top scheme displays the three types of vectors. —Venn diagram representing putative SbGhd7 targets identified by integrating RNA-seq and transient ChIP-seq data; FIG. 2D. —Transient chromatin immunoprecipitation-sequencing (ChIP-seq) read depths in SbFKF1, SbELF3, SbFT10, SbFT1 and SbFT8 loci; FIG. 2E. The 2-kb promoter regions are indicated; FIG. 2F. —Transient transactivation assays show that SbGhd7 targets and represses SbFKF1, SbELF3 and SbFT10, but not SbFT1 or SbFT8. Relative GUS activity (GUS/LUC) was calculated by normalizing GUS activity against luciferase activity Values represent means±SE, n=3. P values were calculated by two-tailed Student's t-tests.

FIGS. 3A-3D—Sorghum SbGhd7 binds to the TGAATG(A/T)(A/T/C) motif. FIGS. 3A-3C. The cis-element TGAATG(A/T)(A/T/C) is prevalent in 2-kb promoter sequences of key flowering genes in crop plants. FIG. 3A—sorghum; FIG. 3B—maize and FIG. 3C—rice; and FIG. 3D. —Electrophoretic mobility shift assay (EMSA) assay showing that SbGhd7 specifically binds to the DNA probe containing three copies of TGAATGAA sequence (probe 1), but not the mutated probe by replacing AT with GG (probe 2).

FIGS. 4A-4B—SbEhd1 activates flowering and SbFTs expression in sorghum. FIG. 4A. —Quantitative RT-PCR results showing SbEhd1 overexpression activates the expression of SbFT1, SbFT8 and SbFT10. Gene expression was normalized against the expression of SbActin, with Ctrl set as 1. Values represent means±SE, n=4. P values were calculated by two-tailed Student's t-tests; and FIG. 4B. —Transactivation assay testing SbEhd1's transcriptional activation on SbFT10, SbFT1, SbFT8 and SbGhd7 promoters. Left scheme displays the used three types of vectors. Relative GUS activity (GUS/LUC) was calculated by normalizing GUS activity against luciferase activity. Values represent means±SE, n=3. P values were calculated by two-tailed Student's t-tests.

FIG. 5 Computational modeling of the interaction between the CCT domain of SbGhd7 and the TGAATGAA DNA helix. Protein and DNA helix were docked manually (Methods). Residues that may be involved in the interaction are shown in sticks. The close-up view (bottom) shows the potential interactions of the AT in the middle of the DNA motif with residues Arg 192, Lys 196, Arg 200 and Tyr 202 in the CCT domain of SbGhd7. Showing, carbon atom; oxygen atom; nitrogen atom; phosphorus atom; dash line-hydrogen bond.

FIGS. 6A-6B—The purification of SbGhd7Δ protein for EMSA assay in FIGS. 3A-3D. FIG. 6A—The prediction using protein disorder prediction server (PrDOS) showing that the region (SbGhd7Δ; aa184-231) containing the CCT domain (aa186-227) has low disorder and is suitable for in vitro protein expression and purification; and FIG. 6B—Coomassie dye-stained SDS-PAGE gel showing purified tag-free SbGhd7Δ proteins (indicated by the arrow).

FIG. 7. Protein sequence alignment of rice OsGhd7 (E5RQA1.1, NCBI) and sorghum SbGhd7. The CCT DNA binding domain is indicated by the arrow.

### DETAILED DESCRIPTION

The present disclosure provides a cis-regulatory element that is recognized and bound by SbGhd7/Ghd7 to precisely reprogram the gene regulatory network controlling crop flowering, which consequently improves biomass production and grain production.

The present cis-regulatory element can be used for precise flowering time manipulation with low side effects. The cis-regulatory element can be removed or mutated in promoters of desired genes using the CRISPER-Cas12a promoter editing system. For example, to precisely manipulate flowering time, the cis-regulatory element within promoter of specific target genes that play a role in plant growth and development can be altered by removing or mutating the cis-regulatory element to reprogram the gene regulatory network to obtain the desired phenotype. This cis-regulatory element, that binds to a transcription factor in target genes to repress flowering, can be removed or mutated to reduce side effects of the transcription factor binding and promote grain production. In addition, this cis-regulatory element can be added to promoters of certain genes to manipulate flowering time to eliminate their negative effects on plant growth while maintaining necessary plant development to increase biomass as result of transcription factor binding to the added cis-regulatory element.

The present cis-regulatory element and techniques related to it have the benefit of being capable of precisely manipulating gene regulatory network with minimum side effects. Each plant transcription factor generally modulates numerous genes. Traditional methods that manipulate the expression of transcription factor may be problematic because of the side effects that may be inevitable due to the large set of transcription factor gene targets. The present method provides a more precise way to manipulate crop flowering with low side effects.

The manipulation of flowering time is a promising strategy for increasing crop biomass or food production. To this end, previous attempts have focused on overexpressing or knocking out key transcription factors (TFs) and flowering targets (FTs) in the gene regulatory network (GRN) that affect floral transition or flowering. Although flowering time may be altered by overexpressing or knocking out key transcription factors regulating floral transition or flowering, there may be adverse side effects associated with it. For example, manipulations of earlier flowering have been associated with hormonal disruptions, growth penalties and a reduction in seed number and size. These side effects appear to be unavoidable due to the wide range of transcription factor targets and the complicated feedback and feedforward loops in the gene regulatory network. To address this, there remains a need for an improved strategy that can precisely manipulate the regulatory association between transcription factor and the desired target genes modulated either directly or indirectly by the selected transcription factor, for example, by modifying a specific cis-regulatory element (e.g. removal or mutation) in the promoter of certain desired target genes that can bind directly by the selected transcription factor.

The present concept may be used in bioenergy and agricultural industries. The cis-regulatory element can be removed or mutated in promoters of desired genes using the CRISPER-Cas12a promoter editing system for crop improvement. For example, to precisely manipulate flowering time, specific genes known to be involved in floral transition that have cis-regulatory element recognized by SbGhd7 can be modified (e.g. removal or mutation) to regulate direct or indirect effects by those specific genes having the cis-regulatory element recognized by SbGhd7. In addition, the cis-regulatory element recognized by SbGhd7 can be added to promoters of genes with negative effects on plant growth to manipulate flowering time while maintaining necessary plant development. Therefore, this innovation provides a solution to precisely manipulate crop flowering time for food and biomass production with better control of the side effects of bioengineering gene regulatory networks.

The present cis-regulatory element and techniques related to it may enable precise and predictable bioengineering and biodesign of crop flowering either to lengthen the vegetative growth period to improve biomass yield and hence bioenergy feedstock production or shorten the vegetative growth period to increase grain yield. The effects of manipulating the cis-regulatory element may be validated in transgenic crops. Transgenic plant crops (e.g., maize, rice and sorghum) may be generated by removing or mutating the cis-regulatory element in the promoters of target genes involved in flowering using genome editing approaches. The flowering time may be recorded to validate the effects of the cis-regulatory element in controlling crop flowering time between wildtype plants and transgenics in which the cis-regulatory element is validated.

In one embodiment, it is contemplated that the cis-regulatory element, TGAATG(A/T)(A/T/C), present within the promoter sequences (−2001 to −1 bp) of SbEhd1 (Sobic.001G227900), OsFTL10/FT8 (LOC_Os05g44180), ZmELF3.2 (Zm00001d039156/ZmPHB47.06G296000), Rice Ghd7 (LOC_Os07g15770), or Maize Ghd7 (Zm00001d024909) can be removed or mutated using the CRISPER-Cas12a promoter editing system to control flowering time.

The transition from vegetative to reproductive growth is a phase change or a major phase change in flowering plants that ensures continuity and survival. Plants synchronize the timing of their flowering with changes in environmental cues such as for example, light and temperature to achieve reproductive success. Photoperiod (day length) is one factor or one of the major environmental factors that control reproductive competence and floral transition (Jackson, 2009). To initiate floral transition, ‘long-day’ (LD) plants require a day length longer than a certain threshold, called critical day length, ‘short-day’ (SD) plants require a day length shorter than the critical daylength, and ‘day-neutral’ plants flower independently of photoperiod (Jackson, 2009). The critical day length varies greatly depending on the plant species and environmental conditions.

### Example

Sorghum, a multipurpose plant crop providing food, feed and biofuel feedstock, is well suited for sustainable agriculture in semiarid regions due to its ability to grow on marginal lands and resiliency to high temperatures. Sorghum is a typical SD plant and its cultivation for grain in temperate regions uses day-neutral genotypes produced either by natural mutation or introgression of one or more day-neutral recessive alleles (Stephens et al., 1967). In the temperate summer, wild type short day sorghums, may reach six meters in height, only transitioning to the reproductive phase near the end of the growing season as day length gets shorter, producing few or no seed. In contrast, day-neutral lines initiate reproductive growth early, often at ˜1 meter in height, producing less vegetation but more grain. Thus, while photoperiod sensitive sorghums are cultivated for forage and biomass yield, photoperiod neutrality favors grain production in temperate regions.

Understanding of the critical importance of photoperiodic control of floral transition in sorghum cultivation, long recognized by the identification of six classical maturity loci named Ma1-Ma6 in which dominant alleles confer photoperiod sensitivity (Quinby, 1966; Roy, 1974; Rooney & Aydin, 1999), is nonetheless in its infancy. Even the molecular identities and/or functions of some of the classical maturity loci remain controversial. For example, Ma1 is the strongest of these loci, replaced by introgression in 89% of ‘converted’ exotic lines and accounting for ˜85.7% of LD flowering time variation between SD and day-neutral genotypes (Lin et al., 1995). First suggested to be PSEUDORESPONSE REGULATOR 37 (SbPRR37), suppressing flowering in LDs (Murphy et al., 2011), subsequent work reported several lines of evidence that Ma1 is FLOWERING LOCUS T (FT) gene SbFT12 (Cuevas et al., 2016). Ma6 corresponds to a CCT (CONSTANS, CO-like, and TOC1) domain-containing protein encoding gene GRAIN NUMBER, PLANT HEIGHT AND HEADING DATE 7 (SbGhd7) (Murphy et al., 2014), whose rice homolog (Ghd7) represses flowering in LDs by suppressing the expression of the transcription activator EARLY HEADING DATE 1 (Ehd1) (Itoh et al., 2010). SbGhd7 was found to have a circadian clock-regulated expression pattern and was associated with LD-induced late flowering in photoperiod-sensitive sorghum accessions (Murphy et al., 2014; Yang et al., 2014). However, the molecular function of SbGhd7 remain to be investigated.

In plants, the mobile florigens are the ultimate inducers of flowering (Putterill & Varkonyi-Gasic, 2016). Sorghum has three putative florigens activating flowering: SbFT1/SbCN15, SbFT8/SbCN12 and SbFT10/SbCN8. SbFT8 and SbFT10 are strongly induced by SD photoperiods in sensitive genotypes and all the three genes are capable of activating flowering in transgenic Arabidopsis, with SbFT10 having the strongest effect (Yang et al., 2014; Wolabu et al., 2016). The FT protein belongs to the phosphatidylethanolamine-binding (PEBP)-related kinase inhibitor family. Previous phylogenetic analysis of plant PEBP proteins revealed that SbFT1 is clustered with rice HEADING DATE 3a (Hd3a), and SbFT8 and SbFT10 are in a separate subclade containing maize Zea mays CENTRORADIALIS 8 (ZCN8) (Wolabu et al., 2016). Hd3a and ZCN8 are well-documented FT/FT-like genes that activate flowering (Kojima et al., 2002; Lazakis et al., 2011; Meng et al., 2011), suggesting that SbFT1, SbFT8 and SbFT10 are possible functional florigens in sorghum. However, has not been confirmed in sorghum.

To understand the molecular mechanism that controls the flowering pathway and uncover the role or quantitative significance of the three florigens in sorghum, phenotypic analysis was performed on transgenic sorghum and characterization of the molecular functions of SbGhd7 was performed using genome-wide and molecular approaches. By identifying and experimentally validating regulatory targets of SbGhd7 and SbEhd1, it was discovered that SbGhd7 directly represses SbEhd1 and SbFT10, and indirectly regulates SbFT1 and SbFT8 by repressing SbEhd1 expression. Besides SbEhd1 and SbFT10, SbGhd7 directly represses FLAVIN-BINDING, KELCH REPEAT, F-BOX1 (SbFKF1) and EARLY FLOWERING 3 (SbELF3), which promote flowering in SD plants (Yang et al., 2013; Han et al., 2015; Lu et al., 2017). Furthermore, it was defined that SbGhd7 binds to the DNA motif TGAATG(A/T)(A/T/C). This motif is distributed in flowering related gene promoters in sorghum, suggesting that SbGhd7 may be a major regulator of floral transition. Overall, this study provides insight into the molecular function of SbGhd7 and its regulatory role in sorghum floral transition.

### Materials and Methods

### Plant Materials and Growth Conditions

In short day (SD) condition, plants were grown with an 8:16 hr light-dark cycle and a temperature of 24-27° C. with 70-80% relative humidity and 150 Imol m2 light intensity.

Sorghum bicolor (L.) Moench genotypes, BTx623 (photoperiod insensitive, ma1) and Tx430 (photoperiod insensitive, ma1) were generously provided by Bill Rooney, and 100M (photoperiod sensitive, Ma1) and Wheatland (photoperiod insensitive, ma1) were obtained from USDA-ARS, GRIN.

### RNA Extraction, RNA-Seq and Data Analysis

Total RNAs were isolated using the TRIzol Reagent (Invitrogen) from leaves of 4-weeks-old sorghum plants (T0 transgenic plants at 4 weeks post sowing out of tissue culture) and treated with DNase I (Invitrogen) to remove genomic DNA contamination. RNA quality was evaluated by 2100 Bioanalyzer (Agilent). For quantitative RT-PCR (qPCR) and semiquantitative RT-PCR, cDNAs were synthesized using the oligo (dT) primer and SuperScript IV reverse transcriptase (Invitrogen) according to the manufacturer's instructions.

### Protoplast Isolation and Transfection

The middle region of leaf blades from 12-15 days-old BTx623 sorghum plants grown in soil was cut into 1-1.5 mm strips and digested in enzymatic solution (0.4 M mannitol, 20 mM of KCl, 20 mM of MES, 1.5% cellulose R10, 0.4% of Macro enzyme R10, 10 mM of CaCl2, 5 mM 2-ME and 0.1% BSA) in the dark at room temperature for 2.5 hours without shaking and 1 hour with gentle shaking. The released protoplasts were collected by 200 g centrifuging and resuspended in ‘MMg’ solution (0.4 M mannitol, 15 mM of MgCl2, and 4 mM of MES) to the final concentration of 1×106 cells/ml for transfection. The PEG-calcium method was used for protoplast transfection (Xie et al., 2020).

### Subcellular Localization and Transient Transactivation Assays in Protoplasts

SbGhd7 and SbEhd1 cDNAs were cloned into a transient expression vector (Xie et al., 2020) for C-terminal YFP fusion, respectively. 8 μg of this plasmid was co-transfected with 2 μg of mCherry-VirD2NLS plasmid (Lee et al., 2008) into 100 μl of protoplast suspension (˜100,000 cells). After 16 hours incubation under weak light at room temperature, protoplasts were collected and imaged using a Leica TCS SP5 confocal microscope, equipped with 488 and 543 nm laser lines for excitation of YFP and mCherry, respectively. The emission bandwidth for YFP and mCherry was 500-530 nm and 580-620 nm, respectively. Images were processed using LAS X software (Leica).

Transient transactivation assays were performed as described (Xie et al., 2020). A total of 10 μg of effector, reporter, and/or transactivator plasmids were co-transfected into 100 μl of protoplast suspension (˜100,000 cells). 100 ng of 35S:luciferase plasmid was co-transfected for each reaction to normalize GUS activity. After 18 to 20 hours incubation in dark at room temperature, protoplasts were collected and lyzed for measuring GUS and luciferase activities using Synergy Neo2 multimode plate reader (BioTek). GUS and luciferase activities were measured as described (Yoo et al., 2007). GUS activity in individual samples was normalized against luciferase activity (GUS/LUC). Three replicates were performed for statistical calculation.

### Transient ChIP-Seq Experiment and Data Analysis

SbGhd7 was fused with 10×Myc tag in a transient expression vector and transfected into 200 μl of sorghum protoplast suspension (˜200,000 cells). After 14 hours incubation at room temperature, protoplasts were collected for western blot for measuring protein expression and μChIP experiment as previously described (Xie et al., 2020). ChIPed DNA and Input DNA were cleaned and concentrated using MinElute PCR Purification Kit (Qiagen). Sequencing libraries were prepared using ThruPLEX DNA-Seq Kit and DNA Unique Dual Index Kit (Takara) following the manufacturer's manual. 16 libraries were pooled for paired-end 100 bp sequencing using the DNBSEQ instrument (BGI). Approximately 20-30 M reads were obtained for each sample.

After filtering to remove adaptor sequences and low-quality reads, clean reads were mapped to the reference genome Sorghum bicolor v3.1.1 (Phytozome) using BWA-MEM package (v0.7.17.2). Mapped data of ChIP samples and Input samples from two biological replicates with >50% correlation was then pooled for peak calling using MACS2 (v2.2.7.1). The ChIPseeker package (v1.18.0) was then used to annotate narrow peaks identified by MACS2.

### DNA Motif Analysis

To predict DNA motifs bound by SbGhd7, 100-bp flanking sequences around the top 100 peak summits before the peak annotation and target identification were analyzed using the MEME-CHIP (Multiple Em for Motif Elicitation) and SEA (Simple Enrichment Analysis) functions of the MEME suite (Bailey et al., 2015).

The occurrence of the TGAATG(A/T)(A/T/C) motif in promoters of floral genes were searched using the FIMO function of the MEME suite (v5.5.2). 2 kb promoter sequences (−2001 to −1 bp) of floral genes were obtained from Phytozome Sorghum bicolor genome (v3.1.1).

### In Vitro Protein Expression and Purification

SbGhd7Δ (aa184-231) was codon optimized and cloned into a modified pET11e expression vector containing a C-terminal TEV (tobacco etch virus) cleavage site followed by a Strep-tag II. Protein was then overexpressed in E. coli BL21 (DE3) cells by autoinduction at 17° C. for 18 hrs (Studier, 2005). Cells were pelleted and resuspended in lysis buffer [50 mM sodium phosphate, pH 7.6, 1 mM TCEP, 1 mg/mL lysozyme, and complete protease inhibitor cocktail (Roche)]. The mixture was then homogenized on ice using a cell disruptor. Insoluble detritus was removed by centrifuging and the desired proteins were purified by cation exchange chromatography [Mono S 5/50 GL (Cytiva); elution with a linear gradient of 0-500 mM NaCl]. Strep-II was then cleaved by TEV protease digestion and tag-free proteins were polished by size-exclusion chromatography [Superdex Increase 75 10/300 GL (Cytiva); 50 mM sodium phosphate, pH 7.6, 150 mM NaCl]. Fractions containing SbGhd7Δ were determined by SDS-PAGE, pooled, and concentrated. To assess the identity and quality of SbGhd7Δ, its molecular weight was approximated with small angle x-ray scattering (National Synchrotron Light Source II; 16-ID) and by size-exclusion chromatography (Superdex 30 Increase 3.2/300 GL with protein standards).

### Electrophoretic Mobility Shift Assay

DNA probes were end labelled with biotin using DNA 30 End Biotinylation Kit (Thermo Scientific) in accordance with the manufacturer's manual. The electrophoretic mobility shift assay was performed using LightShift Chemiluminescent EMSA Kit (Thermo Scientific). 0.5 μg of purified protein and 1 μM of biotin-labeled probe were used and incubated at room temperature for 20 min. For competition assays, 50× or 200× unlabeled DNA probe was added. The reaction mixtures were then resolved in 6% DNA retardation gel (Novex) by electrophoresis at 100 V for 1-2 h and transferred to Nylon membranes. Biotin signals were detected using the Chemiluminescent Nucleic Acid Detection Module (Thermo Scientific) as suggested by the manufacturer.

### Computational Simulation of Protein-DNA Interaction

AlphaFold2 (Jumper et al., 2021) was used to predict the structure of the CCT DNA binding domain of SbGhd7 (Ser 174 to Arg 220). To simulate its DNA binding and define residues involved in the interaction, the helix of TGAATGAA DNA motif and the CCT domain of SbGhd7 were docked manually using COOT (Emsley & Cowtan, 2004) and PYMOL.

Editing of Cis-Regulatory Element that Binds to SbGhd7/Ghd7 in Certain Genes of Interest Using the CRISPR-Cas12a System

The CRISPR-Cas12a system with high genome editing efficiency in monocot plants will be used for the promoter editing (Zhang, Yingxiao, et al, 2021). For each target gene, two CRISPR RNAs (crRNAs) that flank the identified cis-regulatory element(s) in its promoter are designed to delete the promoter region containing the cis-regulatory element(s). The crRNA sequences are listed in Table 1. The T-DNA vectors for transgenic plant generation are constructed as described previously (Zhang, Yingxiao, et al, 2021). Briefly, the two crRNAs are synthesized as duplexed DNA oligonucleotides, annealed and cloned into crRNA expression vectors pYPQ131-STU-Lb and pYPQ132-STU-Lb (Addgene), respectively, using Esp3l enzyme digestion. The two crRNAs are then assembled into the recipient vector pYPQ142-ZmUbi (Addgene) using the Golden Gate reaction. The two crRNAs in this vector together with the cas 12a in the entry vector pYPQ230-RRV (Addgene) are assembled into the T-DNA vector pYPQ203 to get the final T-DNA vector for transgenics. This T-DNA vector is transformed into the destination host plant (e.g. Sorghum bicolor TX430, Oryza sativa Nipponbare, and Zea mays B73) using the Agrobacterium-mediated methods (Ignacimuthu S, et al., 2014, Himizu-Sato S et al., 2020, and Liu, Qiangbo et. al, 2023). DNAs from the regenerated plants from callus will be extracted and sequenced to identify transgenic plants with the desired promoter deletion. The flowering time of the correctly edited transgenic plants will be compared with that of the non-edited plants to validate impacts on flowering regulation.

TABLE 1

| crRNA | | Host | | Target |
|---|---|---|---|---|
| Name | crRNA sequence | Plant | Target Gene | Gene Name |
| crRNA1 | TAACTTGCAATGTCAAGTT | Sorghum | Sobic.001G227900 | SbEhd1 |
| | GGAC (SEQ ID NO: 1) | | | |
| crRNA2 | ATTAAGTCCAAGTTGGATG | Sorghum | Sobic.001G227900 | SbEhd1 |
| | TAAT (SEQ ID NO: 2) | | | |
| crRNA3 | TACTGGAGTTGAGGAAGGC | Sorghum | Sobic.009G199900 | SbFT10 |
| | TGTC (SEQ ID NO: 3) | | | |
| crRNA4 | TGCAACATGTCAGCAACCA | Sorghum | Sobic.009G199900 | SbFT10 |
| | ATCC (SEQ ID NO: 4) | | | |
| crRNA5 | AAAGGTGAATGTACAAGAC | Rice | LOC_Os05g44180 | OsFTL10/ |
| | AACC (SEQ ID NO: 5) | | | FT8 |
| crRNA6 | TATGGTGAATGATGAGGGT | Rice | LOC_Os05g44180 | OsFTL10/ |
| | TGTC (SEQ ID NO: 6) | | | FT8 |
| crRNA7 | TCAAACGGGTACCGGAATG | maize | Zm00001d039156/ | ZmELF3.2 |
| | TCCG (SEQ ID NO: 7) | | ZmPHB47.06G296000 | |
| crRNA8 | ACAAAGTGGCCATTCAGAG | maize | Zm00001d039156/ | ZmELF3.2 |
| | ATCT (SEQ ID NO: 8) | | ZmPHB47.06G296000 | |

### Data Availability

The raw reads data of RNA-seq and transient ChIP-seq have been deposited to Gene Expression Omnibus (GEO) with the access number GSE238095.

### Accession Numbers

Sequence data can be found under the following Phytozome accession numbers (Sorghum bicolor v3.1.1): SbGhd7 (Sobic.006G004400), SbEhd1 (Sobic.001G227900) SbFT1 (Sobic.010G045100), SbFT8 (Sobic.003G295300), SbFT10 (Sobic.009G199900), SbFKF1 (Sobic.005G145300), SbELF3 (Sobic.009G257300), OsFTL10/FT8 (LOC_Os05g44180), ZmELF3.2 (Zm00001d039156/ZmPHB47.06G296000), Rice Ghd7 (LOC_Os07g15770), Maize Ghd7 (Zm00001d024909), and SbActin (Sobic.005G047100).

Genes described herein and/or orthologs thereof may in certain embodiments be exemplified by nucleotide sequences that comprise from about 34%, 40%, 50%, 60%, 62%, 70%, 80%, 85%, 90%, 95% to about 100% sequence identity to sequences described herein, or a complement thereof including SEQ ID Nos herein.

### Results

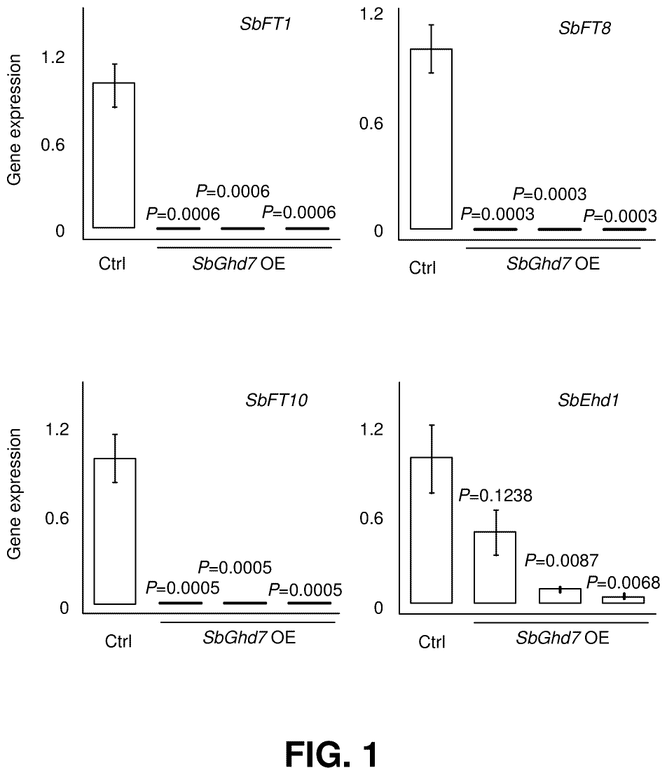

Quantitative RT-qPCR results showed that expressions of functional florigens SbFT1, SbFT8 and SbFT10 were significantly downregulated in SbGhd7 overexpression lines (FIG. 1, Table 2). In addition, the expression of SbEhd1, whose homolog in rice was found to be negatively controlled by Ghd7 (Nemoto et al., 2016), was also downregulated in SbGhd7 overexpression lines (FIG. 1, Table 2). These results demonstrate that SbGhd7 is a key regulator of flowering time in sorghum and negatively controls floral transition pathway.

### SbGhd7 is a Transcriptional Repressor

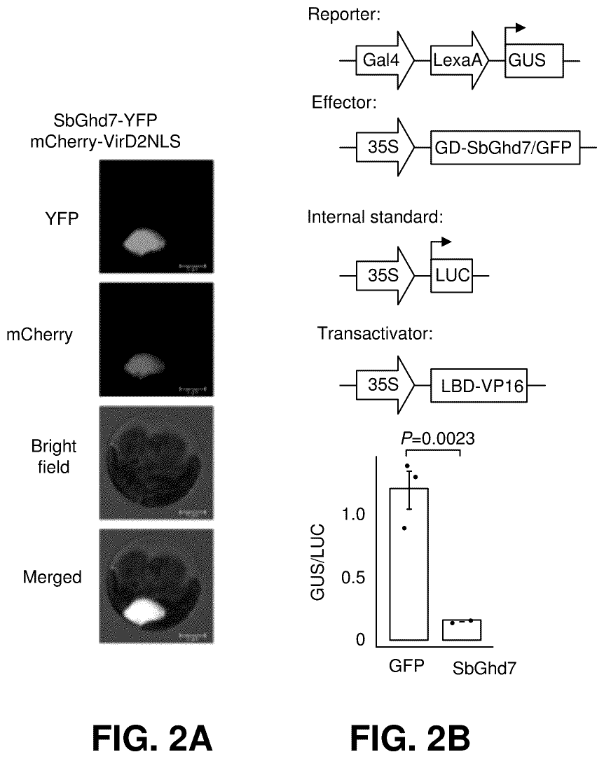

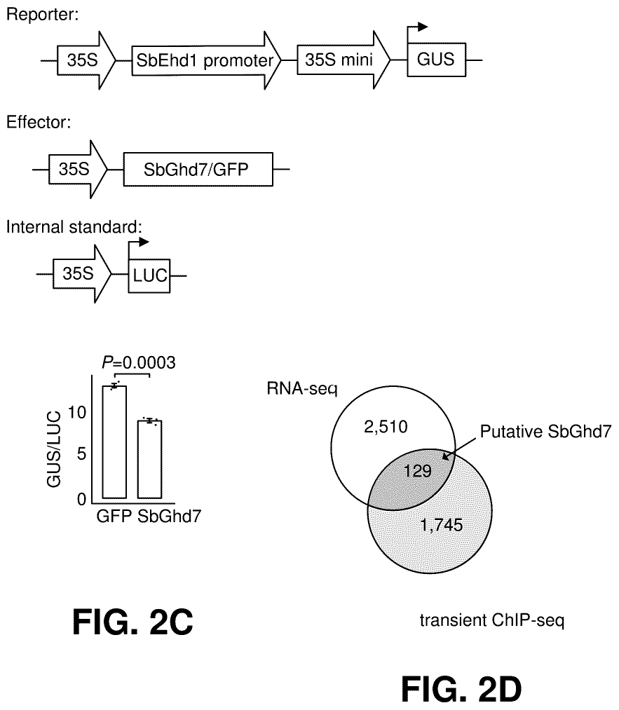

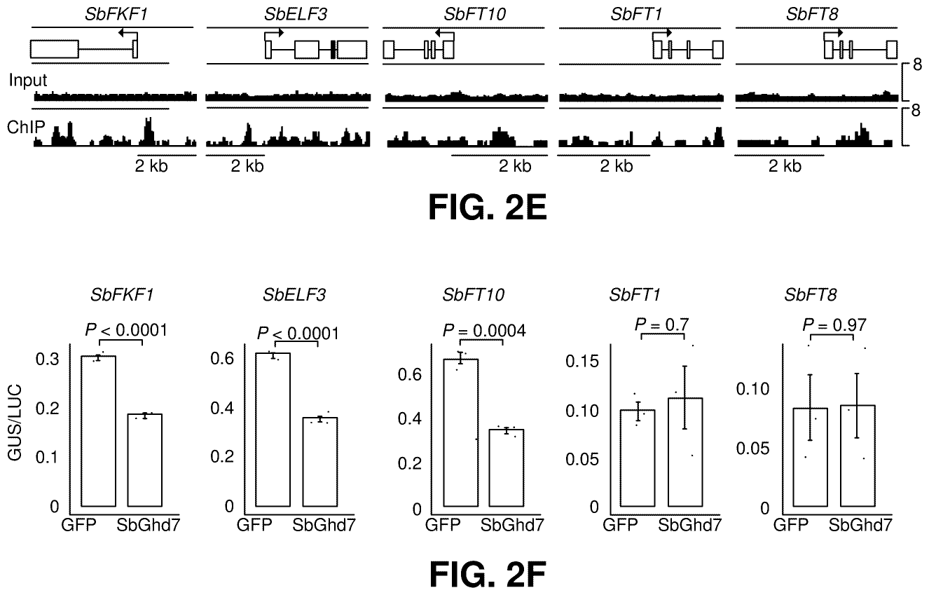

The SbGhd7 protein is localized in the nucleus in sorghum protoplasts (FIG. 2A). Furthermore, protoplast-based transient transactivation assays using the GUS and luciferase reporter system (Xie et al., 2020) revealed that SbGhd7 has transcriptional repressor activity, but not activator activity (FIG. 2B). SbGhd7 was fused with the Gal4 DNA-binding domain (GD-SbGhd7) and recruited to the promoter of the GUS reporter gene via the interaction between GD and the Gal4 DNA sequence upstream of GUS. A plasmid overexpressing luciferase (LUC) was co-transfected as an internal standard. To test repressor activity, an additional transactivator plasmid overexpressing LexA DNA-binding domain (LBD) fused herpes simplex virus VP16 transactivator (VP16) was co-transfected to constitutively activate GUS expression through the interaction between LexA and LBD. The normalized GUS activity (GUS/LUC) was used to evaluate transcriptional activity of SbGhd7. As shown in FIG. 2B, SbGhd7 has a significantly lower GUS/LUC value than the green fluorescent protein (GFP) negative control, indicating transcriptional repressor activity.

Noting that its rice homolog represses the maturity gene Ehd1 (Itoh et al., 2010), SbEhd1 promoter was inserted between the 35S promoter and the GUS reporter gene and co-transfected with a plasmid overexpressing SbGhd7 or the GFP negative control (FIG. 2C). As indicated by the GUS/LUC values, SbGhd7 can specifically repress the activity of the SbEhd1 promoter (FIG. 2C). This shows that SbGhd7 directly targets SbFKF1, SbELF3 and SbFT10.

Analysis of the genome-wide targets of SbGhd7 repression, the transcriptomes of SbGhd7 OE and Ctrl were compared by RNA-seq. This identified 3,880 upregulated and 2,639 downregulated genes by SbGhd7 repression. In transient ChIP-seq experiments, 1,874 genes showed SbGhd7 binding peaks localizing to the 3 kb proximal promoter regions, of which 129 were also downregulated in the RNA-seq (FIG. 2C). These 129 putative SbGhd7 target genes were highly enriched for flowering time genes including blue-light photoreceptor FLAVIN-BINDING, KELCH REPEAT, F-BOX1 (SbFKF1), EARLY FLOWERING 3 (SbELF3), and SbFT10.

Noting significant SbGhd7 binding peaks in the promoters of SbFKF1, SbELF3 and SbFT10 (FIG. 2E), inserting SbFKF1, SbELF3 and SbFT10 promoters between the 35S promoter and the GUS reporter gene, respectively, the transient transactivation assay confirmed that SbGhd7 directly represses the activity of all three promoters (FIG. 2F). While SbFT1, SbFT8 and SbFT10 all activated flowering in transgenic Arabidopsis (Wolabu et al., 2016) and each was significantly downregulated in SbGhd7 overexpression lines (FIG. 1), no SbGhd7 binding peaks or transcriptional repression were detected in the SbFT1 or SbFT8 promoters (FIGS. 2E and 2F), suggesting that their control could be indirect through other regulators.

### SbGhd7 Binds to the DNA Motif TGAATG(A/T)(A/T/C)

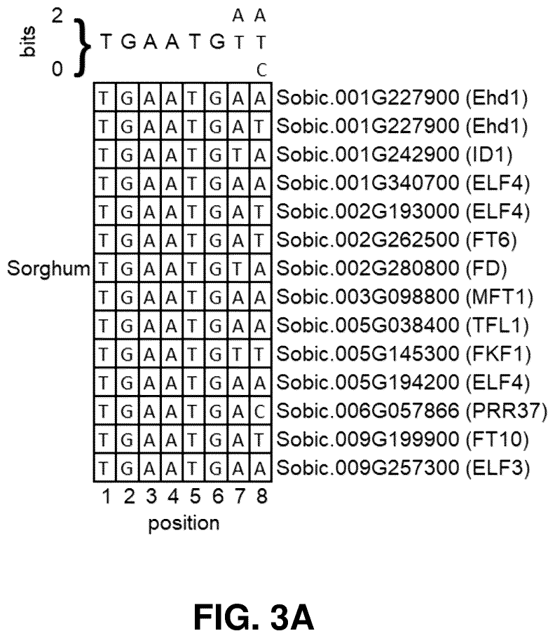

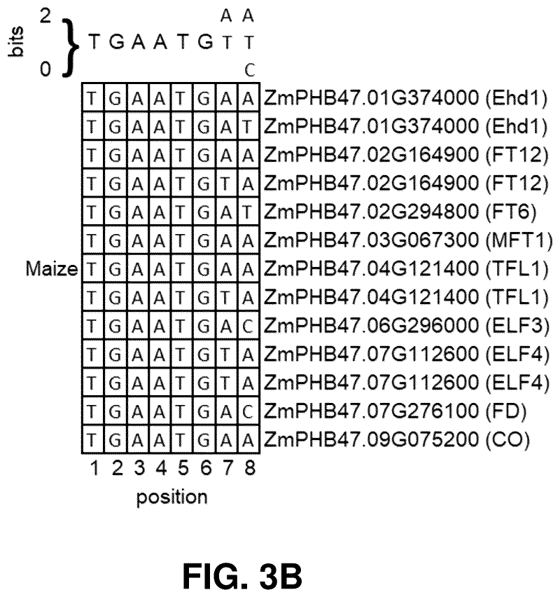

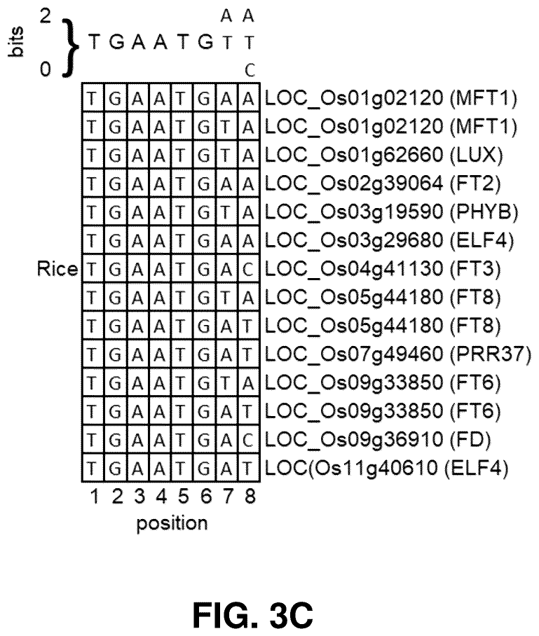

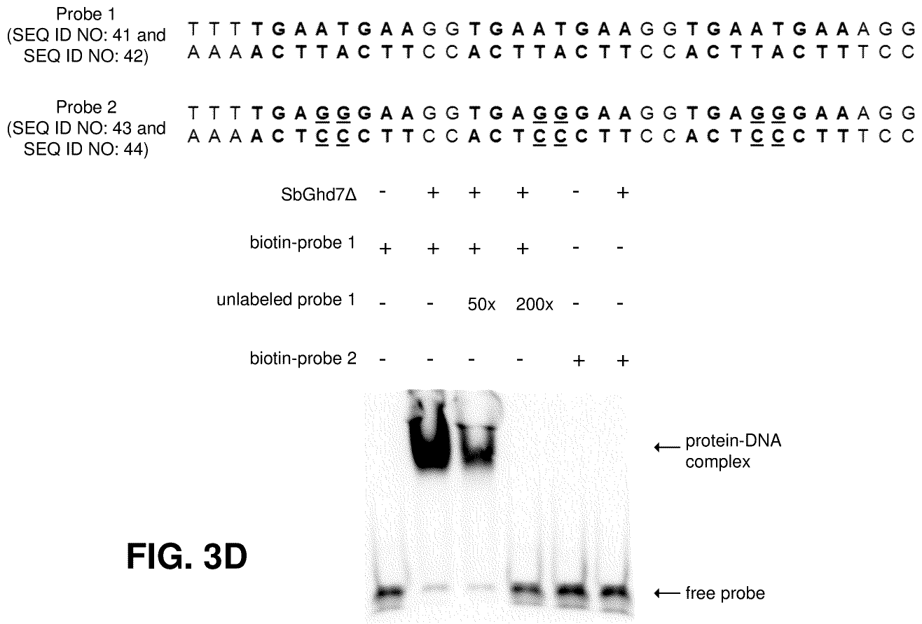

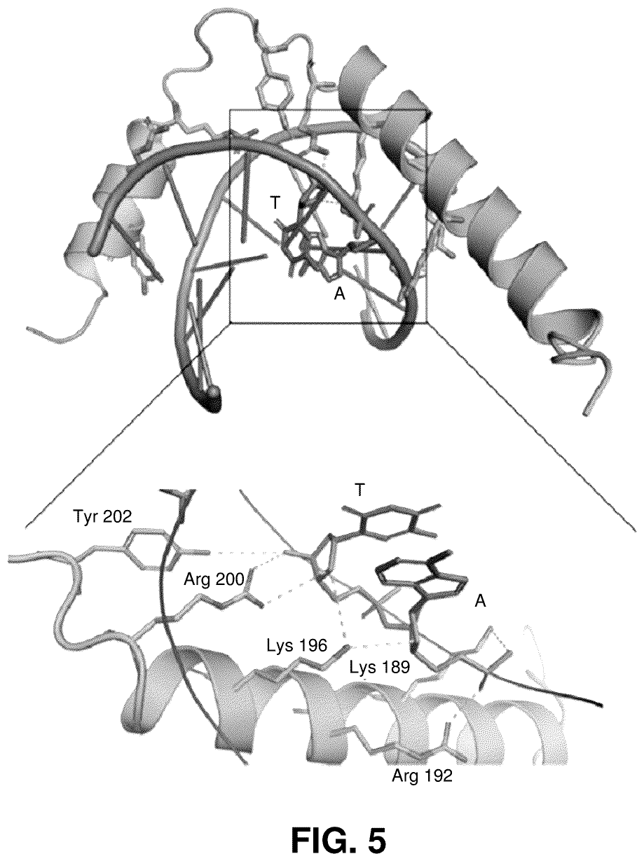

The 100-bp DNA sequences flanking the top 100 SbGhd7 peak summits identified from transient ChIP-seq are significantly enriched for TGAATG(A/T)(A/T/C) sequence (e-value, 2.71×10-7; FIG. 3A and Methods). This motif is present in the 2-kb promoters of 13 out of 31 sorghum floral transition genes including SbEhd1, SbFT10, SbELF3, and SbFKF1 (FIG. 3A). Computational simulation suggests that the nucleotides AT in the middle of the TGAATG(A/T)(A/T/C) motif may be essential for the protein-DNA interaction (FIG. 5).

TABLE 2
Primers used in this study.

| Gene | Gene | Forward Primer | Reverse Primer | |
|---|---|---|---|---|
| ID | Name | (5′ to 3′) | (5′ to 3′) | Purpose |
| Sobic. | SbGhd7 | GGGGACAAGTTTGTACAAAAAA | GGGGACCACTTTGTACAAGAA | Clone |
| 006G004400 | | GCAGGCTTCATGTCAGGGCCAG | AGCTGGGTCTCAGAGCAGGTG | gene |
| | | CATGC (SEQ ID NO: 9) | GTCGTTG (SEQ ID NO: | |
| | | | 10) | |
| Sobic. | SbFT10 | GGGGACAAGTTTGTACAAAAAA | GGGGACCACTTTGTACAAGAA | Clone |
| 009G199900 | | GCAGGCTTCATGTCAGCAACCA | AGCTGGGTCCTACTCTTCCCG | gene |
| | | ATCCTTTGG (SEQ ID NO: | AAACCTTCT (SEQ ID NO: | |
| | | 11) | 12) | |
| Sobic. | SbFT1 | CACCGCTCGACCGTCCTTGACT | GTCGTCGTCCAATCCCTACGA | Clone |
| 010G045100 | | ATCCAA (SEQ ID NO: 13) | CC (SEQ ID NO: 14) | gene |
| Sobic. | SbFT8 | CACCGAAGTGCATTGAAGTGTC | TGTTGGAGAACAAGGACAACT | Clone |
| 003G295300 | | AGAAC (SEQ ID NO: 15) | T (SEQ ID NO: 16) | gene |
| Sobic. | SbFT10 | CACCCTGACAATGGCTATGATC | GTTGCAGAAAAATAACTTGTG | Clone |
| 009G199900 | | TAGAC (SEQ ID NO: 17) | AA (SEQ ID NO: 18) | promoter |
| Sobic. | SbEhd1 | CACCAGGCTTGCTGTAGCTGCA | TCGACCTGATTATCAAGAGAG | Clone |
| 001G227900 | | AAGCGTCCAT (SEQ ID NO: | A (SEQ ID NO: 20) | promoter |
| | | 19) | | |
| Sobic. | SbFT1 | CACCGCTCGACCGTCCTTGACT | GTCGTCGTCCAATCCCTACGA | Clone |
| 010G045100 | | ATCCAA (SEQ ID NO: 21) | CC (SEQ ID NO: 22) | promoter |
| Sobic. | SbFT8 | CACCGAAGTGCATTGAAGTGTC | TGTTGGAGAACAAGGACAACT | Clone |
| 003G295300 | | AGAACT (SEQ ID NO: 23) | T (SEQ ID NO: 24) | promoter |
| Sobic. | SbELF3 | CACCCCTCTTGATTTGCCAAAG | GTCTAGTAGCAGCGGAGGTGG | Clone |
| 009G257300 | | GATCCC (SEQ ID NO: 25) | AGGT (SEQ ID NO: 26) | promoter |
| Sobic. | SbFKF1 | CACCGAAACCTTCTTAACGCTA | ACAGCCTCATCCGCTTCACC | Clone |
| 005G145300 | | TTC (SEQ ID NO: 27) | (SEQ ID NO: 28) | promoter |
| Sobic. | SbActin | TGAAGCGGGTGAGAAGATTGT | AGCCAAATCATACTCGCCCA | qPCR/ |
| 001G048200 | | (SEQ ID NO: 29) | (SEQ ID NO: 30) | RT-PCR |
| Sobic. | SbFT10 | ATGTCAGCAACCAATCCTTT | TAGAAAGCCCTCATGTCATTG | qPCR/ |
| 009G199900 | | (SEQ ID NO: 31) | (SEQ ID NO: 32) | RT-PCR |
| Sobic. | SbEhd1 | TGTGGCCTTCTGGGCTAAGG | TCAGTTGGAAAGCACACATCG | qPCR/ |
| 001G227900 | | (SEQ ID NO: 33) | C (SEQ ID NO: 34) | RT-PCR |
| Sobic. | SbFT1 | CTTCGTCCGGACCACCAACCTC | AGCAGATCACCTCTTGCCCAA | qPCR/ |
| 010G045100 | | A (SEQ ID NO: 35) | ATGCT (SEQ ID NO: 36) | RT-PCR |
| Sobic. | SbFT8 | ATGACTGATGTGGAACCGTT | TGCAAATACTCCCTCAGTGAT | qPCR/ |
| 003G295300 | | (SEQ ID NO: 37) | (SEQ ID NO: 38) | RT-PCR |
| Sobic. | SbGhd7 | TCATTCTGTGGGAGCACATTC | ACACCTCCTCATCCTCTTCTC | qPCR/ |
| 006G004400 | | (SEQ ID NO: 39) | (SEQ ID NO: 40) | RT-PCR |

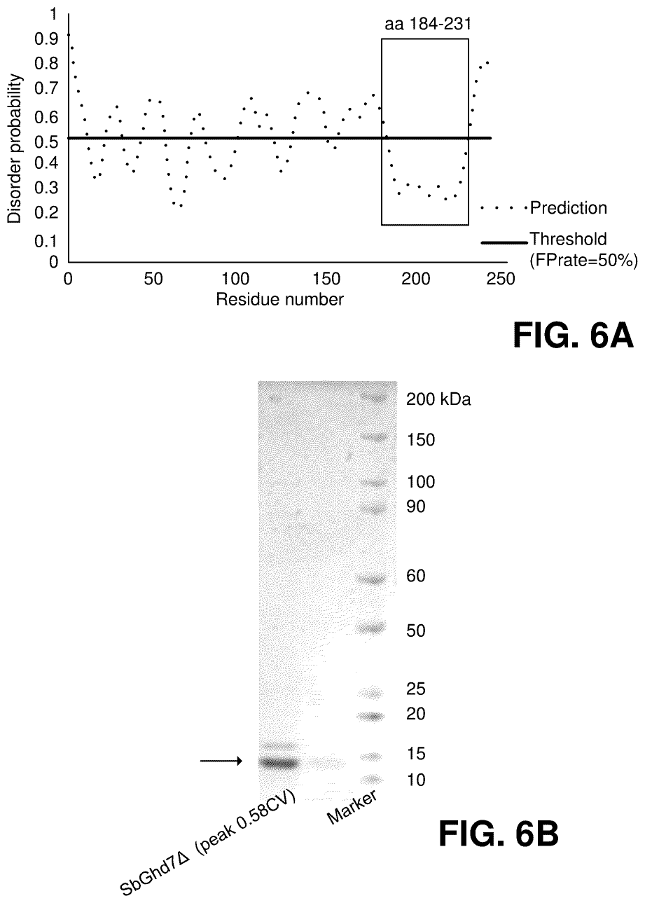

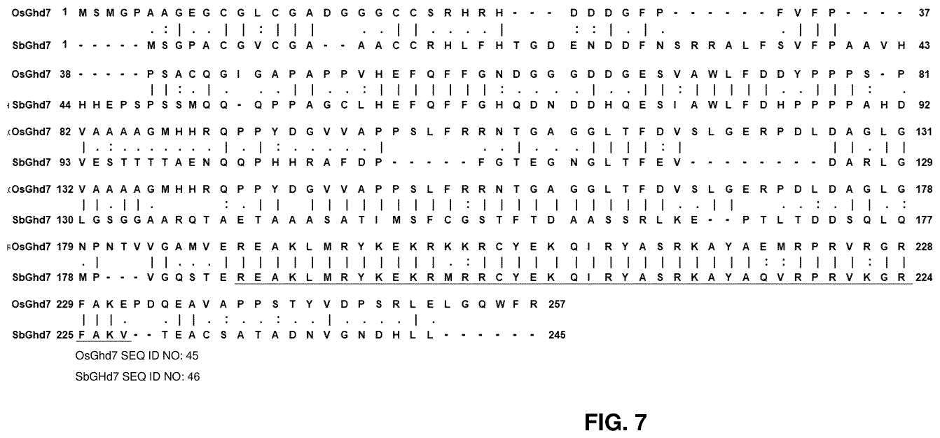

Electrophoretic mobility shift assay (EMSA) confirmed that SbGhd7 binds specifically to this DNA motif (FIG. 3B). We expressed and purified the DNA binding domain of SbGhd7 (SbGhd74; amino acids 184-231; FIG. 6A, 6B) and generated biotin labeled DNA probe containing three copies of the TGAATGAA sequence (probe 1; FIG. 3B). EMSA and the competition assay using 50× and 200× unlabeled probe of the same sequence demonstrated that the binding was specific as indicated by reduction of the bound signal (protein-DNA complex) in the presence of the competitor (FIG. 3B). A mutated probe replacing AT with GG (probe 2; FIG. 3B) showed no binding to SbGhd7, suggesting that the AT is critical for SbGhd7-DNA binding.

### SbEhd1 Induces Early Flowering by Activating SbFTs Expression

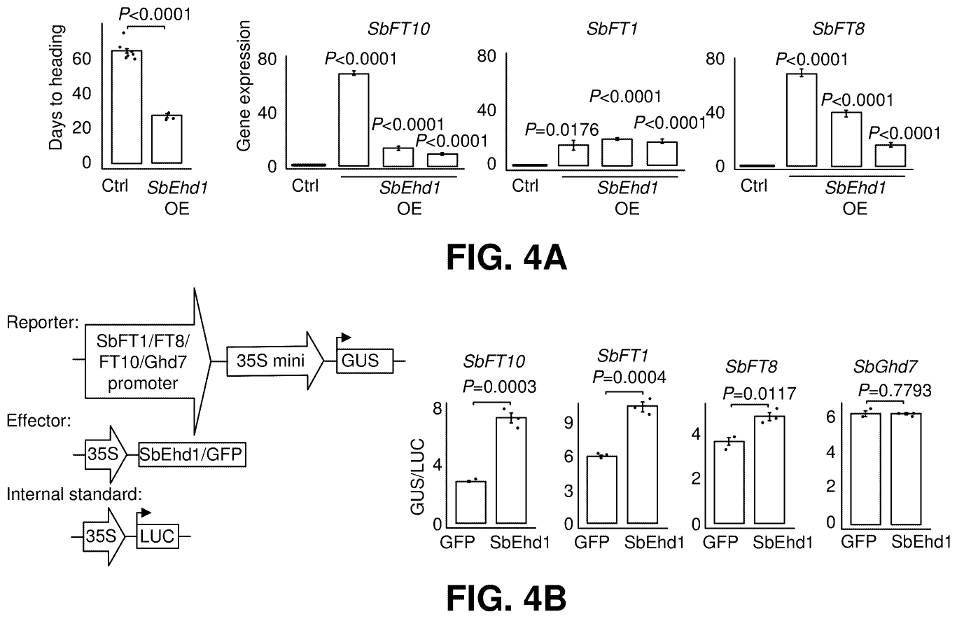

Since SbGhd7 directly targets and represses SbEhd1 expression, we hypothesize that indirect regulation of SbGhd7 on SbFT1 and SbFT8 may occur via repression of SbEhd1 expression. In contrast to the non-flowering of SbGhd7 overexpression lines, SbEhd1 overexpression dramatically accelerated flowering and significantly upregulated the expression of SbFT1, SbFT8 and SbFT10 (FIG. 4A).

Tests with sorghum protoplasts revealed that SbEhd1 is localized in the nucleus and acts as a transcription activator. Using the transient transactivation assay, each SbFT promoter was inserted in front of the GUS reporter gene in the reporter constructs and co-transfected with the construct overexpressing SbEhd1 or GFP control. SbEhd1 activated the promoters of SbFT1, SbFT8 and SbFT10, but not SbGhd7 (FIG. 4B), indicating direct downstream activation of SbFT expression.

The central importance of the floral transition for reproductive fitness may have conferred a sustained selective advantage to the establishment or maintenance of multiple, seemingly redundant signals. The more striking phenotypic consequences of SbGhd7 overexpression than its rice ortholog OsGhd7, appear to reflect a combination of direct regulation of the major florigen SbFT10, reinforced by additional florigens SbFT1 and SbFT8 that are activated by SbEhd1). SbFT8 and SbFT10 are paleologs, and are among only 14.2% of genes for which both copies remain both in sorghum and rice (OsFTL9, OsFTL10) (Paterson et al., 2009) since duplicating in a common ancestor ˜98 million years ago (Wang et al., 2015). Floral induction by SbFT10 and its reinforcement by SbFT8 (via SbEhd1) appears ancient, and its repression by SbGhd7 may impart permanent vegetative growth. On the other hand, OsGhd7 is believed to be a key component of the rice external coincidence model that explains the relationship between photoperiod and flowering time control (Vicentini et al., 2023). The mRNA and protein accumulation of OsGhd7 highly depend on photoperiod. SDs have been found to not only reduce OsGhd7 transcription but also reduce OsGhd7 protein stability via post-translational mechanisms, even when it is overexpressed (Zheng et al., 2019). Although SbGhd7 transcription is controlled by SDs, SbGhd7 overexpression completely blocked sorghum flowering, suggesting that the post-translational mechanism controlling Ghd7 protein accumulation in SDs may be absent in sorghum. This is possible considering the divergent evolution of these two monocot crops in very different environments. Another aspect of their divergent evolution is indicated by the differences in cis-element binding of OsGhd7 and SbGhd7. OsGhd7 was found to bind to the CACA/TGTG sequence, which is absent from the OsEhd1 promoter (Nemoto et al., 2016). Therefore, it was hypothesized that OsGhd7 represses OsEhd1 through the formation of a protein complex instead of direct promoter binding (Nemoto et al., 2016). In contrast, the present study found that SbGhd7 binds to the TGAATG(A/T)(A/T/C) sequence, which widely presents in promoters of sorghum flowering genes including SbEhd1. Since the CCT DNA binding domain of OsGhd7 and SbGhd7 is highly conserved, it is possible thatOsGhd7 is capable of binding to the TGAATG(A/T)(A/T/C) motif.

Scrutiny of SbGhd7 reveals new dimensions of floral regulation, perhaps reflecting botanical diversity or alternatively merely easier to resolve due to its striking effects. In the facultative LD plant Arabidopsis, the blue-light receptor FKF1 promotes flowering in LDs and provides timing information for CONSTANS (CO) stabilization (Imaizumi et al., 2003; Imaizumi et al., 2005; Song et al., 2012). In the facultative SD plant rice, OsFKF1 promotes flowering independent of day length by activating Ehd1, Ehd2 and repressing Ghd7 (Han et al., 2015). In seeming contrast, SbGhd7 is an upstream negative regulator of SbFKF1 (FIGS. 2E and 2F). An intriguing hypothesis is whether a feedback loop may exist whereby SbGhd7 represses SbFKF1 in LDs and SbFKF1 represses SbGhd7 independent of photoperiod to promote flowering, which might be tested by quantifying SbGhd7 expression in SbFKF1 overexpression and knockout lines.

Similarly, SbELF3, a homolog of Arabidopsis ELF3, was found to be directly regulated by SbGhd7 (FIG. 2F). In Arabidopsis, ELF3 interacts with the red light photoreceptor PHYB to mediate photoperiodic flowering (McWatters et al., 2000; Liu et al., 2001; Kolmos et al., 2011), together with EARLY FLOWERING 4 (ELF4) and LUX ARRHYTHMO (LUX) forming the evening complex (EC) that delays floral transition under noninductive SD photoperiods (Nusinow et al., 2011). ELF3 homologs promote flowering of rice (OsELF3) under LD conditions by negatively regulating OsGI and OsGhd7 (Yang et al., 2013); and of soybean (GmELF3) in SD photoperiods by directly repressing the legume-specific flowering repressor E1 (Lu et al., 2017). QTL and genome-wide association studies have linked SbELF3 to flowering time (Li et al., 2018; Hu et al., 2019), but how it affects floral transition remains to be elucidated.

The relationship between SbGhd7 and Ma1, on the same chromosome and thought to be the major classical maturity locus inhibiting LD flowering, remains unknown. Although currently depicted downstream of PHYB (Yang et al., 2014), it remains unclear whether LD upregulation of SbGhd7 transcription is a direct response to light or an indirect response through photoreceptors and other light sensors. The SbGhd7-encoded CCT domain-containing protein is not known to bind to or be associated with chromophores, nor is it likely to perceive photons directly, as it lacks protein motifs common to known photoreceptors, including phytochrome, cryptochrome, phototropin, xanthopsin, blue light sensor using FAD (BLUF) and rhodopsin (Moglich et al., 2010). It could amplify and transduce light signals by interactions downstream of photon perception, although this has not yet been investigated.

A direct relationship with rice phytochromes OsPHYA and OsPHYB stabilizes the Ghd7 protein post-transcriptionally and delays flowering (Zheng et al., 2019) in wild-type genotypes but not in phytochrome mutants. The Ghd7 overexpression effect was reduced by coexpression with rice GIGANTIA (OsGI), which destabilizes the Ghd7 protein (Zheng et al., 2019).

In summary, the study demonstrates that SbGhd7 is a major repressor of sorghum flowering and expands the molecular genetic understanding of the regulation of sorghum floral transition. The SbGhd7-centric gene regulatory network controlling sorghum floral transition provides opportunities to bioengineer this key trait for sorghum improvement and provides a fundamental framework for studying the regulation of sorghum flowering time in response to environmental cues.

### Data Availability

The data that support the findings of this study are openly available in Gene Expression Omnibus (GEO), reference number GSE238095.

### Definitions

“Mutation” for reducing gene expression includes deletion, insertion and/or substitution of one or more nucleotides of the gene of sequences regulating expression of the gene. In one embodiment, said mutation comprises, deleting at least a portion of sequences that regulates transcription of the gene, introducing deletion, insertion and/or substitution, etc. so that at least one mutated allele contains an altered portion of the promoter region. In one embodiment, said deleted sequences may be replaced with polynucleotides that are exogenous to the deleted gene sequences and that are flanked by sequences that are complementary to polynucleotide regions of the endogenous gene that flank the deleted gene sequences. In a further embodiment, at least one mutated allele is generated by site specific recombination, frame shift mutation, homologous recombination, CRISPR gene editing, or any combination thereof, in a cell such as an embryonic stem cell or germ cell.

“Transformation” is a process of introducing a DNA sequence or construct (e.g., a vector or expression cassette) into a cell or protoplast in which that exogenous DNA is incorporated into a chromosome or is capable of autonomous replication.

“Transgenic” and “genetically engineered” cell or plant refers to a cell or plant comprising cells that have a genome that has been manipulated by any molecular biological technique, including, for example, the introduction of a transgene, homologous recombination, knockin of a gene, knockout of a gene, and/or CRISPR gene editing.

The term “transgene” refers to any nucleic acid sequence that is introduced into the cell by experimental manipulations. A transgene may be an “endogenous” DNA sequence or a “heterologous DNA sequence.”

“Endogenous” molecule (such as nucleotide sequence, amino acid sequence, cis-regulatory element) is a molecule natively found in nature in a host cell or a cell of the same species.

“Heterologous” and “foreign” molecule (such as nucleotide sequence, amino acid sequence, cis-regulatory element) is a molecule that is not endogenous. In one embodiment, a heterologous sequence contains some modification (e.g., mutation, the presence of a selectable marker gene, etc.) relative to the naturally-occurring sequence. In this respect, the heterologous sequence may be native to the host genome, but be rearranged with respect to other genetic sequences within the host sequence. For example, a regulatory sequence may be heterologous in that it is linked to a different coding sequence relative to the native regulatory sequence. In addition, a particular sequence can be “heterologous” with respect to a cell or organism into which it is introduced (for example, a sequence that does not naturally occur in that particular cell or organism).

The term “control plant,” as used herein, refers to a plant or crop plant of the same species that does not comprise the modification or modifications described in this disclosure. In some embodiments, the control plant or is of the same variety. In some embodiments, the control plant is of the same genetic background.

Plant or “crop plant” refers to a plant cell and/or tissue and/or organ, exemplified by flower, and other plant parts such as the seed, leaf, pollen, ovule, fruit, rootstock, and scion. In one embodiment, the crop plant comprises a flower produced by the presently disclosed methods.

The term “DNA,” as used herein, refers to a nucleic acid molecule of one or more nucleotides in length. By “nucleotide” it is meant a naturally-occurring nucleotide, as well as modified versions thereof. The term “DNA” includes double-stranded DNA, single-stranded DNA, isolated DNA such as cDNA, as well as modified DNA that differs from naturally-occurring DNA by the addition, deletion, removal, substitution, manipulation, mutation, modification and/or alteration of one or more nucleotides as described herein.

The term “expression” refers to the process of converting genetic information of a polynucleotide into RNA through transcription, which is catalyzed by an enzyme, RNA polymerase and into protein, through translation of mRNA on ribosomes. Expression can be, for example, constitutive or regulated, such as, by up-regulation or overexpression refers to regulation that increases the production of expression products (mRNA, polypeptide or both) relative to basal or native states, while inhibition or down-regulation refers to regulation that decreases production of expression products (mRNA, polypeptide or both) relative to basal or native states. Expression of a gene can be measured through a suitable assay, such as real-time quantitative reverse transcription polymerase chain reaction (qRT-PCR), Northern blot, transcriptome sequencing and Western blot.

The term “gene,” as used herein, refers to a segment of nucleic acid that encodes an individual protein or RNA and can include both exons and introns together with associated regulatory regions such as promoters, operators, terminators, 5′ untranslated regions, 3′ untranslated regions, and the like.

The terms “alter” “edit” and “modify” when in reference to the level of any molecule (e.g., amino acid sequence, and nucleic acid sequence, etc.) and/or phenomenon (e.g., level of expression of a gene, level of transcription of a DNA sequence, level of translation of an mRNA molecule to an amino acid sequence) and/or phenotype (e.g., flowering rate, flowering time, grain production, biomass production) in a first composition (e.g., first crop plant) relative to a second composition (e.g., second crop plant), mean that the quantity of flowering and/or phenomenon and/or phenotype in the first composition refer to an increase and/or decrease in the level of flowering and/or phenomenon and/or phenotype.

A transgenic crop plant is disclosed herein that comprises a genomically changed sequence of a cis-regulatory element in the promoter of certain genes which alters the flowering time of the plant. The cis-regulatory element in the promoter of such certain genes may be modified from TGAATG(A/T)(A/T/C) to TGAGGG(A/T)(A/T/C). Preferably the genes which alters the flowering time of the plant are genes that encode SbEhd1, OsFTL10/FT8, SbFT10, ZmELF3.2, or combinations thereof. Preferably the crop plant is sorghum, maize or rice. More preferably, the transgenic crop plant is sorghum. In an embodiment, the present transgenic crop plant is a progeny plant. Flowering time of the transgenic crop plant can be regulated by the present method of changing the sequence of the cis-regulatory element in the promoter of certain genes.

### REFERENCES

Bailey T L, Johnson J, Grant C E, Noble W S. 2015. The MEME suite. Nucleic acids research 43(W1): W39-W49.
Cuevas H E, Zhou C, Tang H, Khadke P P, Das S, Lin Y R, Ge Z, Clemente T, Upadhyaya H D, Hash C T, et al. 2016. The Evolution of Photoperiod-Insensitive Flowering in Sorghum, A Genomic Model for Panicoid Grasses. Mol Biol Evol 33(9): 2417-2428.
Emsley P, Cowtan K. 2004. Coot: model-building tools for molecular graphics. Acta Crystallogr D Biol Crystallogr 60(Pt 12 Pt 1): 2126-2132.
Han S H, Yoo S C, Lee B D, An G, Paek N C. 2015. Rice FLAVIN-BINDING, KELCH REPEAT, F-BOX 1 (OsFKF 1) promotes flowering independent of photoperiod. Plant, Cell & Environment 38(12): 2527-2540.
Hu Z, Olatoye M O, Marla S, Morris G P. 2019. An Integrated Genotyping-by-Sequencing Polymorphism Map for Over 10,000 Sorghum Genotypes. Plant Genome 12(1): 180044.
2005. FKF1 F-box protein mediates cyclic degradation of a repressor of CONSTANS in Arabidopsis. Science 309(5732): 293-297.
Imaizumi T, Tran H G, Swartz T E, Briggs W R, Kay S A. 2003. FKF1 is essential for photoperiodic-specific light signalling in Arabidopsis. Nature 426(6964): 302-306.
Itoh H, Nonoue Y, Yano M, Izawa T. 2010. A pair of floral regulators sets critical day length for Hd3a florigen expression in rice. Nat Genet 42(7): 635-638.
Jackson S D. 2009. Plant responses to photoperiod. New Phytol 181 (3): 517-531.
Jumper J, Evans R, Pritzel A, Green T, Figurnov M, Ronneberger O, Tunyasuvunakool K, Bates R, Zidek A, Potapenko A, et al. 2021. Highly accurate protein structure prediction with AlphaFold. Nature 596 (7873): 583-589.
Kojima S, Takahashi Y, Kobayashi Y, Monna L, Sasaki T, Araki T, Yano M. 2002. Hd3a, a rice ortholog of the Arabidopsis FT gene, promotes transition to flowering downstream of Hd1 under short-day conditions. Plant Cell Physiol 43(10): 1096-1105.
Kolmos E, Herrero E, Bujdoso N, Millar A J, Toth R, Gyula P, Nagy F, Davis S J. 2011. A reduced-function allele reveals that EARLY FLOWERING3 repressive action on the circadian clock is modulated by phytochrome signals in Arabidopsis. Plant Cell 23(9): 3230-3246.
Lazakis C M, Coneva V, Colasanti J. 2011. ZCN8 encodes a potential orthologue of Arabidopsis FT florigen that integrates both endogenous and photoperiod flowering signals in maize. J Exp Bot 62(14): 4833-4842.
Lee L Y, Fang M J, Kuang L Y, Gelvin S B. 2008. Vectors for multi-color bimolecular fluorescence complementation to investigate protein-protein interactions in living plant cells. Plant Methods 4:24.
Li X, Guo T, Mu Q, Li X, Yu J. 2018. Genomic and environmental determinants and their interplay underlying phenotypic plasticity. Proc Natl Acad Sci USA 115(26): 6679-6684.
Lin Y R, Schertz K F, Paterson A H. 1995. Comparative analysis of QTLs affecting plant height and maturity across the Poaceae, in reference to an interspecific sorghum population. Genetics 141(1): 391-411.
Liu G, Godwin I D. 2012. Highly efficient sorghum transformation. Plant Cell Rep 31(6): 999-1007.
Liu X L, Covington M F, Fankhauser C, Chory J, Wagner D R. 2001. ELF3 encodes a circadian clock-regulated nuclear protein that functions in an Arabidopsis PHYB signal transduction pathway. Plant Cell 13(6): 1293-1304.
Lu S, Zhao X, Hu Y, Liu S, Nan H, Li X, Fang C, Cao D, Shi X, Kong L, et al. 2017. Natural variation at the soybean J locus improves adaptation to the tropics and enhances yield. Nat Genet 49(5): 773-779.
McWatters H G, Bastow R M, Hall A, Millar A J. 2000. The ELF3 zeitnehmer regulates light signalling to the circadian clock. Nature 408(6813): 716-720.
Meng X, Muszynski M G, Danilevskaya O N. 2011. The FT-like ZCN8 Gene Functions as a Floral Activator and Is Involved in Photoperiod Sensitivity in Maize. Plant Cell 23(3): 942-960.
Moglich A, Yang X, Ayers R A, Moffat K. 2010. Structure and function of plant photoreceptors. Annu Rev Plant Biol 61:21-47.
Murphy R L, Klein R R, Morishige D T, Brady J A, Rooney W L, Miller F R, Dugas D V, Klein P E, Mullet J E. 2011. Coincident light and clock regulation of pseudoresponse regulator protein 37 (PRR37) controls photoperiodic flowering in sorghum. Proc Natl Acad Sci USA 108 (39): 16469-16474.
Murphy R L, Morishige D T, Brady J A, Rooney W L, Yang S, Klein P E, Mullet J E. 2014. Ghd7 (Ma6) Represses Sorghum Flowering in Long Days: Ghd7 Alleles Enhance Biomass Accumulation and Grain Production. The Plant Genome 7(2).
Nemoto Y, Nonoue Y, Yano M, Izawa T. 2016. Hd1, a CONSTANS ortholog in rice, functions as an Ehd1 repressor through interaction with monocot-specific CCT-domain protein Ghd7. Plant J 86(3): 221-233.
Nusinow D A, Helfer A, Hamilton E E, King J J, Imaizumi T, Schultz T F, Farre E M, Kay S A. 2011. The ELF4-ELF3-LUX complex links the circadian clock to diurnal control of hypocotyl growth. Nature 475(7356): 398-402.
Paterson A H, Bowers J E, Bruggmann R, Dubchak I, Grimwood J, Gundlach H, Haberer G, Hellsten U, Mitros T, Poliakov A, et al. 2009. The Sorghum bicolor genome and the diversification of grasses. Nature 457(7229): 551-556.
Putterill J, Varkonyi-Gasic E. 2016. FT and florigen long-distance flowering control in plants. Current Opinion in Plant Biology 33:77-82.
Quinby J. 1966. Fourth Maturity Gene Locus in Sorghum 1. Crop Science 6(6): 516-518.
Rooney W L, Aydin S. 1999. Genetic control of a photoperiod-sensitive response in Sorghum bicolor (L.) Moench. Crop Science 39(2): 397-400.
Roy Q. 1974. Sorghum improvement and the genetics of growth: Texas A&M University Press.
Song Y H, Smith R W, To B J, Millar A J, Imaizumi T. 2012. FKF1 conveys timing information for CONSTANS stabilization in photoperiodic flowering. Science 336(6084): 1045-1049.
Stephens J, Miller F, Rosenow D. 1967. Conversion of alien sorghums to early combine genotypes 1. Crop Science 7(4): 396-396.
Studier F W. 2005. Protein production by auto-induction in high-density shaking cultures. Protein expression and purification 41(1): 207-234.
Tang X, Lowder L G, Zhang T, Malzahn A A, Zheng X, Voytas D F, Zhong Z, Chen Y, Ren Q, Li Q. 2017. A CRISPR-Cpf1 system for efficient genome editing and transcriptional repression in plants. Nature plants 3(3): 1-5.
Vicentini G, Biancucci M, Mineri L, Chirivì D, Giaume F, Miao Y, Kyozuka J, Brambilla V, Betti C, Fornara F. 2023. Environmental control of rice flowering time. Plant communications.
Wang X, Wang J, Jin D, Guo H, Lee T H, Liu T, Paterson A H. 2015. Genome Alignment Spanning Major Poaceae Lineages Reveals Heterogeneous Evolutionary Rates and Alters Inferred Dates for Key Evolutionary Events. Molecular Plant 8(6): 885-898.
Wolabu T W, Tadege M. 2016. Photoperiod response and floral transition in sorghum. Plant Signal Behav 11(12): e1261232.
Wolabu T W, Zhang F, Niu L, Kalve S, Bhatnagar-Mathur P, Muszynski M G, Tadege M. 2016. Three FLOWERING LOCUS T-like genes function as potential florigens and mediate photoperiod response in sorghum. New Phytol 210(3): 946-959.
Xie M, Zhang J, Yao T, Bryan A C, Pu Y, Labbe J, Pelletier D A, Engle N, Morrell-Falvey J L, Schmutz J, et al. 2020. Arabidopsis C-terminal binding protein ANGUSTIFOLIA modulates transcriptional co-regulation of MYB46 and WRKY33. New Phytol 228(5): 1627-1639.
Yang S, Murphy R L, Morishige D T, Klein P E, Rooney W L, Mullet J E. 2014. Sorghum phytochrome B inhibits flowering in long days by activating expression of SbPRR37 and SbGHD7, repressors of SbEHD1, SbCN8 and SbCN12. PLOS One 9(8): e105352.
Yang Y, Peng Q, Chen G X, Li X H, Wu C Y. 2013. OsELF3 is involved in circadian clock regulation for promoting flowering under long-day conditions in rice. Molecular Plant 6(1): 202-215.
Yoo S D, Cho Y H, Sheen J. 2007. Arabidopsis mesophyll protoplasts: a versatile cell system for transient gene expression analysis. Nat Protoc 2(7): 1565-1572.
Zheng T, Sun J, Zhou S, Chen S, Lu J, Cui S, Tian Y, Zhang H, Cai M, Zhu S. 2019. Post-transcriptional regulation of Ghd7 protein stability by phytochrome and Os GI in photoperiodic control of flowering in rice. New Phytologist 224(1): 306-320.
Blumel, M., Dally, N., & Jung, C. Flowering time regulation in crops—what did we learn from Arabidopsis? Current opinion in biotechnology 2015, 32, 121-129.
Century, K., Reuber, T. L., & Ratcliffe, O. J. Regulating the regulators: the future prospects for transcription-factor-based agricultural biotechnology products. Plant physiology 2008, 147(1), 20-29.
Ding, Y., Zhu, J., Zhao, D., Liu, Q., Yang, Q., & Zhang, T. Targeting cis-regulatory elements for rice grain quality improvement. Frontiers in Plant Science 2021, 12, 705834.
Tadesse, D., Yee, E F., Xie, M. & Tadege, M. Sorghum SbGhd7 is a major regulator of floral transition and directly represses genes crucial for flowering activation. New Phytologist 2024. https://doi.org/10.1111/nph.19591. In press.
Zhou, J., Liu, G., Zhao, Y., Zhang, R., Tang, X., Li, L., . . . & Zhang, Y. An efficient CRISPR-Cas 12a promoter editing system for crop improvement. Nature Plants 2023, 9(4), 588-604.
Zhang, Yingxiao, et al. “Expanding the scope of plant genome engineering with Cas 12a orthologs and highly multiplexable editing systems.” Nature communications 12.1(2021): 1944.
Ignacimuthu S, et al. (2014) Development of transgenic Sorghum bicolor (L.) Moench resistant to the Chilo partellus (Swinhoe) through Agrobacterium-mediated transformation. Molecular Biology & Genetic Engineering 2 (1): 1-8.
Himizu-Sato S, Tsuda K, Nosaka-Takahashi M, et al. Agrobacterium-Mediated Genetic Transformation of Wild Oryza Species Using Immature Embryos. Rice (N Y). 2020 Jun. 3; 13(1): 33.
Liu, Qiangbo, Xian Sheng Zhang, and Ying Hua Su. “Application of Wox2a in transformation of recalcitrant maize genotypes.” aBIOTECH (2023): 1-3.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. A transgenic crop plant that comprises: a genomically changed sequence of a cis-regulatory element in the promoter of genes which alters the flowering time of the plant.

2. The transgenic crop plant of claim 1, wherein the cis-regulatory element in the promoter of genes which alters the flowering time of the plant is modified from TGAATG(A/T) (A/T/C) to TGAGGG(A/T) (A/T/C).

3. The transgenic crop plant of claim 1, wherein the genes which alters the flowering time of the plant encode SbEhd1, OsFTL10/FT8, SbFT10, ZmELF3.2, or combinations thereof.

4. The transgenic crop plant of claim 1, wherein the plant is sorghum, maize or rice.

5. The transgenic crop plant of claim 1, wherein said plant is sorghum.

6. A progeny plant of the transgenic plant of claim 1.

7. A method of regulating the flowering time of a transgenic crop plant by changing the sequence of a cis-regulatory element in the promoter of genes which alters the flowering time of the plant.

8. The method of claim 7 wherein the cis-regulatory element is modified from TGAATG(A/T) (A/T/C) to TGAGGG(A/T) (A/T/C).

9. The method of claim 7 wherein the plant is sorghum, maize or rice.

10. The method of claim 7 wherein the plant is sorghum.


SEQUENCE LISTING (46 sequences)

SEQ ID NO: 1 - DNA, 23 bp, synthetic construct
  source 1..23: mol_type = other DNA
taacttgcaatgtcaagttggac

SEQ ID NO: 2 - DNA, 23 bp, synthetic construct
  source 1..23: mol_type = other DNA
attaagtccaagttggatgtaat

SEQ ID NO: 3 - DNA, 23 bp, synthetic construct
  source 1..23: mol_type = other DNA
tactggagttgaggaaggctgtc

SEQ ID NO: 4 - DNA, 23 bp, synthetic construct
  source 1..23: mol_type = other DNA
tgcaacatgtcagcaaccaatcc

SEQ ID NO: 5 - DNA, 23 bp, synthetic construct
  source 1..23: mol_type = other DNA
aaaggtgaatgtacaagacaacc

SEQ ID NO: 6 - DNA, 23 bp, synthetic construct
  source 1..23: mol_type = other DNA
tatggtgaatgatgagggttgtc

SEQ ID NO: 7 - DNA, 23 bp, synthetic construct
  source 1..23: mol_type = other DNA
tcaaacgggtaccggaatgtccg

SEQ ID NO: 8 - DNA, 23 bp, synthetic construct
  source 1..23: mol_type = other DNA
acaaagtggccattcagagatct

SEQ ID NO: 9 - DNA, 27 bp, synthetic construct
  source 1..27: mol_type = other DNA
gcaggcttcatgtcagggccagcatgc

SEQ ID NO: 10 - DNA, 49 bp, synthetic construct
  source 1..49: mol_type = other DNA
ggggaccactttgtacaagaaagctgggtctcagagcaggtggtcgttg

SEQ ID NO: 11 - DNA, 53 bp, Synthetic construct
  source 1..53: mol_type = other DNA
ggggacaagtttgtacaaaaaagcaggcttcatgtcagcaaccaatcctttgg

SEQ ID NO: 12 - DNA, 51 bp, Synthetic construct
  source 1..51: mol_type = other DNA
ggggaccactttgtacaagaaagctgggtcctactcttcccgaaaccttct

SEQ ID NO: 13 - DNA, 28 bp, Synthetic construct
  source 1..28: mol_type = other DNA
caccgctcgaccgtccttgactatccaa

SEQ ID NO: 14 - DNA, 23 bp, synthetic construct
  source 1..23: mol_type = other DNA
gtcgtcgtccaatccctacgacc

SEQ ID NO: 15 - DNA, 27 bp, synthetic construct
  source 1..27: mol_type = other DNA
caccgaagtgcattgaagtgtcagaac

SEQ ID NO: 16 - DNA, 22 bp, synthetic construct
  source 1..22: mol_type = other DNA
tgttggagaacaaggacaactt

SEQ ID NO: 17 - DNA, 27 bp, synthetic construct
  source 1..27: mol_type = other DNA
caccctgacaatggctatgatctagac

SEQ ID NO: 18 - DNA, 23 bp, synthetic construct
  source 1..23: mol_type = other DNA
gttgcagaaaaataacttgtgaa

SEQ ID NO: 19 - DNA, 32 bp, synthetic construct
  source 1..32: mol_type = other DNA
caccaggcttgctgtagctgcaaagcgtccat

SEQ ID NO: 20 - DNA, 22 bp, synthetic construct
  source 1..22: mol_type = other DNA
tcgacctgattatcaagagaga

SEQ ID NO: 21 - DNA, 28 bp, synthetic construct
  source 1..28: mol_type = other DNA
caccgctcgaccgtccttgactatccaa

SEQ ID NO: 22 - DNA, 23 bp, synthetic construct
  source 1..23: mol_type = other DNA
gtcgtcgtccaatccctacgacc

SEQ ID NO: 23 - DNA, 28 bp, synthetic construct
  source 1..28: mol_type = other DNA
caccgaagtgcattgaagtgtcagaact

SEQ ID NO: 24 - DNA, 22 bp, synthetic construct
  source 1..22: mol_type = other DNA
tgttggagaacaaggacaactt

SEQ ID NO: 25 - DNA, 28 bp, synthetic construct
  source 1..28: mol_type = other DNA
cacccctcttgatttgccaaaggatccc

SEQ ID NO: 26 - DNA, 25 bp, synthetic construct
  source 1..25: mol_type = other DNA
gtctagtagcagcggaggtggaggt

SEQ ID NO: 27 - DNA, 25 bp, synthetic construct
  source 1..25: mol_type = other DNA
caccgaaaccttcttaacgctattc

SEQ ID NO: 28 - DNA, 20 bp, synthetic construct
  source 1..20: mol_type = other DNA
acagcctcatccgcttcacc

SEQ ID NO: 29 - DNA, 21 bp, synthetic construct
  source 1..21: mol_type = other DNA
tgaagcgggtgagaagattgt

SEQ ID NO: 30 - DNA, 20 bp, Synthetic construct
  source 1..20: mol_type = other DNA
agccaaatcatactcgccca

SEQ ID NO: 31 - DNA, 20 bp, synthetic construct
  source 1..20: mol_type = other DNA
atgtcagcaaccaatccttt

SEQ ID NO: 32 - DNA, 21 bp, synthetic construct
  source 1..21: mol_type = other DNA
tagaaagccctcatgtcattg

SEQ ID NO: 33 - DNA, 20 bp, Synthetic construct
  source 1..20: mol_type = other DNA
tgtggccttctgggctaagg

SEQ ID NO: 34 - DNA, 22 bp, synthetic construct
  source 1..22: mol_type = other DNA
tcagttggaaagcacacatcgc

SEQ ID NO: 35 - DNA, 23 bp, synthetic construct
  source 1..23: mol_type = other DNA
cttcgtccggaccaccaacctca

SEQ ID NO: 36 - DNA, 26 bp, synthetic construct
  source 1..26: mol_type = other DNA
agcagatcacctcttgcccaaatgct

SEQ ID NO: 37 - DNA, 20 bp, synthetic construct
  source 1..20: mol_type = other DNA
atgactgatgtggaaccgtt

SEQ ID NO: 38 - DNA, 21 bp, synthetic construct
  source 1..21: mol_type = other DNA
tgcaaatactccctcagtgat

SEQ ID NO: 39 - DNA, 21 bp, synthetic construct
  source 1..21: mol_type = other DNA
tcattctgtgggagcacattc

SEQ ID NO: 40 - DNA, 21 bp, Synthetic construct
  source 1..21: mol_type = other DNA
acacctcctcatcctcttctc

SEQ ID NO: 41 - DNA, 34 bp, synthetic construct
  source 1..34: mol_type = other DNA
ttttgaatgaaggtgaatgaaggtgaatgaaagg

SEQ ID NO: 42 - DNA, 34 bp, synthetic construct
  source 1..34: mol_type = other DNA
cctttcattcaccttcattcaccttcattcaaaa

SEQ ID NO: 43 - DNA, 34 bp, Synthetic construct
  source 1..34: mol_type = other DNA
ttttgagggaaggtgagggaaggtgagggaaagg

SEQ ID NO: 44 - DNA, 34 bp, synthetic construct
  source 1..34: mol_type = other DNA
cctttccctcaccttccctcaccttccctcaaaa

SEQ ID NO: 45 - protein, 260 aa, Oryza sativa
MSMGPAAGEGCGLCGADGGGCCSRHRHDDDGFPFVFPPSACQGIGAPAPPVHEFQFFGNDGGGDDGESVAWLFDDYPPPSPVAAAAGMHHRQPPYDGVVAPPSLFRRNTGAGGLTFDVSLGERPDLDAGLGVAAAAGMHHRQPPYDGVVAPPSLFRRNTGAGGLTFDVSLGERPDLDAGLGNPNTVVGAMVEREAKLMRYKEKRKKRCYEKQIRYASRKAYAEMRPRVRGRFAKEPDQEAVAPPSTYVDPSRLELGQWFR

SEQ ID NO: 46 - protein, 245 aa, Sorghum bicolor
MSGPACGVCGAAACCRHLFHTGDENDDFNSRRALFSVFPAAVHHHEPSPSSMQQQPPAGCLHEFQFFGHQDNDDHQESIAWLFDHPPPPAHDVESTTTTAENQQPHHRAFDPFGTEGNGLTFEVDARLGLGSGGAARQTAETAAASATIMSFCGSTFTDAASSRLKEPTLTDDSQLQMPVGQSTEREAKLMRYKEKRMRRCYEKQIRYASRKAYAQVRPRVKGRFAKVTEACSATADNVGNDHLL